(19) United States
(12) Patent Application Publication
(10) Pub. No.: US 2014/0170674 A1
(43) Pub. Date: Jun. 19, 2014

(54) Membraine-Based Assay Devices Utilizing Time-Resolved Up-Converting Luminescence

(71) Applicant: Aimin He, Alpharetta, GA (US)

(72) Inventor: Aimin He, Alpharetta, GA (US)

(21) Appl. No.: 13/730,805

(22) Filed: Dec. 28, 2012

Related U.S. Application Data
(60) Provisional application No. 61/582,425, filed on Jan. 2, 2012.

(51) Int. Cl. G01N 33/558; G01N 33/543

(52) U.S. Cl. CPC G01N 33/558; G01N 33/543
USPC 435/7.4; 436/501; 422/69; 435/287.7

(57) ABSTRACT

This invention discloses an immunochromatographic assay device to detect and quantify analytes. The device utilizes up-converting luminescent probes to detect time-resolved luminescence signals. Because the unconverting luminescent probes can have relatively long emission lifetime, background interference from sample autofluorescence and light scattering from excitation source can be easily eliminated through delayed luminescence detection. Furthermore, the up-converting luminescent probes can be excited by near Infrared (IR) or IR light sources. In comparing with UV and visible lights, the near IR and IR lights can penetrate deeper into sample matrices and more effectively excite the probes, but not the sample matrices, resulting in less background and higher detection sensitivity. A simple and low cost reader can be designed to measure the delayed up-converting luminescence of long lifetime that does not use expensive optical filters and mirrors.

This application claims the benefit under 35 USC §119 (e) of U.S. provisional application Ser. No. 61/582,425 filed Jan. 2, 2012, incorporated herein by reference in its entirety.

### BACKGROUND OF THE INVENTION

Membrane-based lateral flow immunoassays (LFA) have been commercialized for a large number of analytes. Examples of medical diagnostic products that are based on lateral flow immunoassay technology platform include pregnancy tests and drug-of-abuse tests. Currently, those tests are primarily targeting point of care (POC) and over the counter (OTC) test markets because of its low cost and user-friendliness. Most of existing commercial products using the platform provide qualitative and semi-quantitative results. Recently, several emerging lateral flow assay technologies have developed that are capable of providing quantitative measurements. Those technologies use a number of detection techniques in combination with lateral flow assay formats. The reported detection techniques include absorbance or reflectance; conventional fluorescence, phosphorescence, time-resolved fluorescence and phosphorescence, magnetic field, surface-enhance Raman, and up-converting fluorescence. The keys for those different detection techniques are using different types of probes that can be measured. For instance, colored particles such as gold particles and dyed latex particles are used for absorbance or reflectance measurement. Fluorescent particles such as quantum dots and latex particles encapsulated with fluorescent dyes are used as probes for fluorescence measurements. Phosphorescent particles with long emission lifetime have been reported to be used for lateral flow immunoassays to quantify analytes using time-resolved luminescence measurements. Semiconductor nanoparticles are often used as probes for surface-enhanced Raman measurements on lateral flow assays. Magnetic particles have also been used as probes to combine with lateral flow immunoassays for ananlyte quantification by measuring the magnetic field of the particles. Although all those detection techniques have their own merits, none of them are perfect in terms of detection performance and cost. For examples, absorbance or reflectance-based LFA technologies may be relatively simple and cheap to make. They suffer significant drawbacks of low sensitivity. In addition, the color-based detection technologies can not detect blood-based samples and needs sample processing to remove red blood cells before measurement. Conventional fluorescence-based lateral flow assays have been demonstrated to be sensitive. Yet the technologies are rather complexed and expensive because of needs for expensive optical components such as band-pass filters and mirrors. Surface-Raman based lateral low assays systems are also rather complicated and expensive. The detection sensitivity of surface-Raman based lateral low assays systems has been reported to be good and it may provide better specificity as well because of the low background. The magnetic field-based LFA technologies provides good sensitivity and specificity. However, the detection zones and calibration zones of lateral flow device have to be naked to the detector. This is not convenient and subject to contamination because the reagents and devices are not completely sealed from users. Time-resolve fluorescence lateral flow has also been recently reported to provide good sensitivity and specificity. The system is reported to be cheaper than other lateral low detection systems such as conventional fluorescence and surface-enhanced Raman. However, only a limited number of probes is available that is suitable for the detection platform. Those probes often need UV or near UV excitation sources that may often interfere with the analytes and sample matrices such as blood samples.

More recently, up-converting phosphor particles have been developed to be integrated into lateral flow assay devices for highly sensitive detection of analytes. The advantage of this detection platform includes potential high detection sensitivity because of minimal background from samples matrices, and deeper penetration of the IR excitation into tissues and other sample matrices. One biggest shortcoming of this platform is difficulty in obtaining highly uniform and surface functionalized unconverting phosphor nanoparticles. Another shortcoming of this platform is a need to use a powerful excitation IR laser and expensive optical filters to separate the visible fluorescence from the intense IR laser excitation. This combination makes it impractical to use transmissional mode for fluorescence measurement, a preferred mode of measurement to simplify the reader and reduce the cost in comparing with a reflecting measurement mode.

As such, a need currently exists for a simple, inexpensive, user-friendly and sensitive membrane-based assay system for analyte quantification.

### SUMMARY OF THE INVENTION

One embodiment of the present invention is a lateral flow assay device for detecting and quantifying an analyte, comprising: (1) a porous membrane laminated on a solid support material. The supporting material is substantially transparent to visible light, and the porous membrane has a detection zone and a calibration zone physically separated along the liquid flow direction; (2) a first specific binding species immobilized on the detection zone; (3) a second specific species immobilized on the calibration zone; (4) a conjugate pad that is in fluid communication with the porous membrane and is deposited with a detection conjugate and a calibration conjugate that are releasable upon in contact with a liquid sample. The detection conjugate and calibration conjugate have an up-converting fluorescence probes modified with a third and fourth specific binding species, respectively; (5) a wicking pad that collects the fluid that passes through the detection and calibration zone.

Another embodiment of the present invention is a method for detecting and quantifying an analyte, comprising: (1) providing a lateral assay device with a detection zone utilizing an up-converting fluorescent probe having a fluorescence emission lifetime of greater than about 1 microsecond; (2) applying the lateral flow device with the sample; (3) allowing the sample to mix with the up-converting probes and flow to the detection zone; (4) measuring the time-resolved up-converting fluorescence signal of the probes captured on the detection zone by a time-resolved fluorescence reader that comprises a pulsed near IR or IR excitation source and a time-gated detector; (5) comparing the measured signal with a standard curve to obtain the concentration of the analyte in the sample.

The up-converting fluorescent probe may include lanthanide-doped upconversion nanocrystals of samarium, dysprosium, europium, terbium, or combinations thereof. The up-converting fluorescent probe may also include certain chelates of lanthanides of samarium, dysprosium, europium, terbium, or combinations thereof. The up-converting fluorescent probe may further include microparticles and nanoparticles encapsulated with certain chelates and lanthanide-doped upconversion nanocrystals of lanthanides of samarium, dysprosium, europium, terbium, or combinations thereof. Moreover, the up-converting fluorescent probe may have an emission lifetime of greater than 10 microseconds, in some embodiments greater than 50 microseconds, and in some embodiments, from 100 to about 1000 microseconds. Likewise, the up-converting fluorescent probe may have a reversed Stokes shift of greater than 50 nanometers, in some embodiments greater than 100 nanometers, and in some embodiments, from 250 to about 350 nanometers. The probe may be modified with a specific binding species for the analyte.

Another embodiment of the present invention is a method for detecting and quantifying an analyte, comprising: (1) providing a lateral flow assay device with a detection zone and a calibration zone. The device comprises a porous membrane in fluid communication with up-converting fluorescent probes having a fluorescence emission lifetime of greater than 20 microseconds and a reversed Stokes shift greater than 100 nanometers; (2) applying the device with a sample; (3) allowing the sample to mix with the up-converting probes and flow to the detection zone and calibration zone; (4) measuring the time-resolved up-converting fluorescence signals of the probes captured on the detection zone and calibration zone by a time-resolved fluorescence reader that comprises a pulsed near IR or IR excitation source and a time-gated detector; (5) comparing the intensity of the detection signal to the calibration signal, wherein the amount of the analyte is proportional to the intensity of the detection signal calibrated by the intensity of the calibration signal.

The up-converting fluorescent probes at the detection zone may be excited simultaneously or separately from the up-converting fluorescent label at the calibration zone. Likewise, the detection signal and the calibration signal may also be measured simultaneously or separately.

### BRIEF DESCRIPTION OF THE DRAWINGS

FIG. 1 is a perspective view of one embodiment of a membrane-based device of the present invention;

FIG. 2 is a schematic diagram of transmissional measurement mode.

FIG. 3 is a schematic diagram of reflectance measurement mode.

FIG. 4: Scanning electron micrograph of the up-converting luminescence particles obtained in Example 1

FIG. 5 is a graph of time-resolved up-converting excitation and emission spectra of the conjugated luminescence probes obtained in Example 2.

FIG. 6 is the up-converting emission decay for the conjugated probes obtained in Example 2.

### DETAILED DESCRIPTION OF REPRESENTATIVE EMBODIMENTS

### Detailed Description

The present invention discloses a lateral flow assay device for detecting and quantifying an analyte in a sample. The device utilizes time-resolved up-converting luminescence to detect the signals by exciting up-converting luminescence probes using pulsed near IR or IR photons. Because the probes can be excited in near IR region and have a long emission lifetime, background interference from many sources, such as scattered light and autofluorescence, can be practically eliminated during detection. In addition, the luminescence reader used in the present invention can have a simple and inexpensive design. For instance, the reader can utilize a near IR or IR light-emitting diode (LED) for pulsed excitation and a silicon photodiode for accurately detecting delayed luminescence on a membrane-based assay device without using expensive optical components such as monochromators or band-pass optical filters.

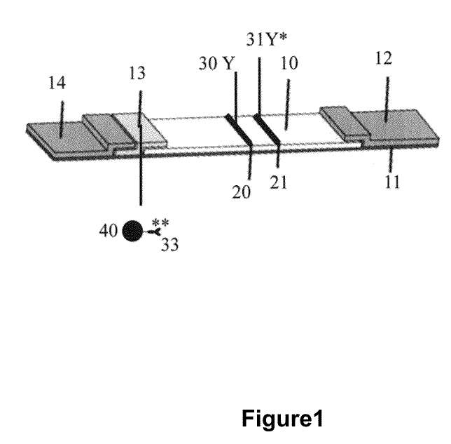

FIG. 1 show a typical lateral flow assay device that comprises a porous membrane 10 laminated on supporting substrate 11. The supporting material may or may not be significantly translucent to UV and/or visible and/or near IR or IR lights. The porous membrane 10 can be selected from a variety of materials through which the test sample is capable of passing through capillary action. For instance, the porous membrane can include, but are not limited to, natural and synthetic, materials such as cellulose materials, paper and cellulose derivatives, cellulose acetate and nitrocellulose, polyether sulfone and nylon membranes. One preferred porous membrane is made of nitrocellulose and/or polyester sulfone materials. The porous membrane may define a detection zone 20 where one or more specific binding species 30 are immobilized. The porous membrane may further define another zone, a calibration zone 21, where one or more specific binding species 31 are immobilized.

The lateral flow assay device may also contain a wicking pad 12. The wicking pad is used to receive fluid that has migrated through the entire porous membrane through capillary action. Typically, the wicking material used to make the wicking pad can include, but not limited to, natural and synthetic, materials such as cellulose materials, paper and cellulose derivatives, cellulose acetate and nitrocellulose.

The lateral flow assay device may also contain a conjugate pad 13 where the up-converting luminescence probes conjugated with one or more specific binding species 33 either covalently or physically (conjugated probes 40) are deposited, and are capable of being released and suspended upon in contact with a liquid sample. The conjugate pad includes, but not limited to, glass fiber pads.

The lateral flow assay device may further comprise a sample pad 14 where a sample is applied. The sample pad can be made of natural and synthetic, materials such as cellulose materials, paper and cellulose derivatives, cellulose acetate and nitrocellulose.

The sample pad 14, conjugate pad 13, porous membrane 10, and wicking pad 12 are typically laminated with a certain amount of overlap so that those components are in fluid communication with each other.

To conduct an assay, a sample may first mix with conjugated probes, and then apply on the porous membrane of a lateral flow device with a detection zone. In this case, the lateral flow device may not need a sample pad and a conjugate pad. The mixture then flows through the detection zone to interact with the specific binding species to form a detection line, which can be measured by a time-resolved fluorescence reader.

When the lateral low device has a sample pad and conjugate pad, the sample may be directly applied to the sample pad 14, the sample then flows to the conjugate pad 13 where the conjugated probes are released and suspended from the conjugate pad into the flow liquid. The sample liquid along with the conjugated probes further flows to the detection zone 20, and calibration zone 21, and finally to the wicking pad 12. Although only one conjugate pad 13 is shown, it should be understood that other conjugate pads may also be used in the present invention. To facilitate accurate detection of a specific analyte, conjugated up-converting luminescence probes may be applied at various locations of the device. The conjugated probes may be used for both detection of the analyte and for calibration. In general, such up-converting luminescence probes can be lanthalide-doped nancrystals, lanthanide chelates, particles encapsulated with lanthalide-doped nancrystals, lanthanide chelates.

The up-converting luminescence probes are configured to allow up-converting excitation by near IR and IR lights and “time-resolved luminescence detection.” The up-converting excitation involves absorption of two or more photons simultaneously by the probe to the excited state and emitting of one photon of higher energy or shorter wavelength than the excitation photons. In this manner, normally long wavelength excitation lights such as near IR and IR photons can be used to excite the probe that can minimize the negative influence of autofluorescence and background. Time-resolved luminescence measurement involves exciting the up-converting luminescence probes with a short pulse of light, then typically waiting a certain time (e.g., between approximately 20 to 200 microseconds) after excitation before measuring the remaining long-lived luminescence signal. In this manner, any short-lived fluorescent background signals and scattered excitation radiation are eliminated. Combination of up-converting excitation and time-resolved luminescence detection can eliminate much of the background signals that can provide sensitivities of a few orders greater than conventional fluorescence. Thus, up-converting time-resolved luminescence detection can reduce background signals from the emission source or from scattering processes (resulting from scattering of the excitation radiation) by taking advantage of the luminescence characteristics of certain up-converting luminescence materials. In addition, the near IR and IR excitation photons are capable of penetrating the porous membrane much deeper than visible and UV photons to excite most or all the up-converting luminescence probes. Using the up-converting luminescence probes for time-resolved luminescence lateral flow assays is much more sensitive than other existing detection techniques. Because visible and UV excitation photons can only penetrate very shallow layer of the porous membranes such as nitrocellulose membranes, only a small portion of the labels are actually excited, resulting in non-optimal detection sensitivity.

One selection criterion of particularly desired probes for time-resolved up-converting luminescence lateral flow assay includes relatively high up-converting efficiency. The high up-converting efficiency will provide strong signal and high detection sensitivity. Using the up-converting probes allows use of low cost and powerful near IR and IR LEDs and lasers. Further benefit of using up-converting fluorescence probes is minimal interference of samples, particularly biological matrices that are normally much more transparent to near IR and IR than UV and visible lights. For instance, biological samples are normally subjected to less damage from near IR or IR lights than UV and visible lights. In addition, the up-converting luminescence probes usually have a relatively large reversed “Stokes shift.” The term “reversed Stokes shift” is referred as the displacement of spectral lines or bands of luminescent radiation to a shorter emission wavelength than the excitation lines or bands. A relatively large reversed Stokes shift allows the excitation wavelength of the up-converting luminescence probe to remain far apart from its emission wavelengths and is desirable because a large difference between excitation and emission wavelengths makes it easier to eliminate the reflected excitation radiation from the emitted signal. Further, excitation of the up-converting luminescence probes by near IR and IR generates minimal autofluorescence from sample matrices, such as proteins and tissues. In addition, a large reversed Stokes shift also minimizes the requirement for expensive, high-precision filters to eliminate background interference. For example, the up-converting luminescence probes have a reversed Stokes shift of greater than 50 nanometers, in some embodiments greater than 100 nanometers, and in some embodiments, from 250 to about 350 nanometers.

Another selection criterion of particularly desired probes for time-resolved up-converting luminescence lateral flow assays includes a relatively long emission lifetime. The long luminescence lifetime is desired so that the probe emits its signal well after any short-lived background signals dissipate. Furthermore, a long luminescence lifetime makes it possible to use low-cost circuitry for time-gated luminescence measurements. For example, luminescence probes used in the present invention may have a luminescence lifetime of greater than 1 microsecond, in some embodiments greater than 10 microseconds, in some embodiments greater than 50 microseconds, and in some embodiments, from 100 microseconds to about 1000 microseconds.

Another selection criterion of particularly desired probes for time-resolved up-converting luminescence lateral flow assays is easy modification of the probes that allows attachment of specific binding species. The attachment can be physical absorption of binding species or covalent bonding. This is important to provide good detection specificity and reagent stability.

One type of luminescence probes that is suitable for time-resolved up-converting luminescence lateral flow assays are doped nanocrystals of lanthanides, and lanthanidee chelates of samarium (Sm (III)), dysprosium (Dy (III)), europium (Eu (III)), and terbium (Tb (III)). Another type of suitable probes is micro- and nano-particles that encapsulate with those chelates and/or doped nanocrystals. Another class of suitable luminescence probes is nanocrystals and aggregates of silver-gold-ligand clusters or particles encapsulated with those aggregates and nanocrystals. Those probes have relatively high up-converting luminescence efficiency, long luminescence lifetime, and are excitable effectively by near IR and IR photons. Furthermore, the reversed Stoke shift for those probes are also large, normally more than 100 nm.

The luminescence probes may be used in a variety of forms. The probes may be used in forms of polymers, liposomes, dendrimers, and other micro- or nano-scale structures. The probes may also be in forms of microparticles and nanoparticles. For example, in one embodiment, latex microparticles that are encapsulated with lanthalide chelates, or doped nanocrystals. The probes are preferred to have functional groups that allow covalent attachment of specific binding species. The functional groups include an aldehyde, carboxyl, amino, hydroxyl, or hydrazide derivative thereof.

When particles are utilized, the particle size may vary depending on factors such as the type of particle chosen, the pore size of the membrane, and the membrane composition. The diameter of the particulate probes can range from 0.01 microns to 1,000 microns, in some embodiments from 0.01 microns to 100 microns, and in some embodiments, from 0.01 microns to 10 microns. In one particular embodiment, the particles have a mean diameter of from 0.1 to 2 microns. Generally, the particles are substantially spherical in shape, although other shapes including, but not limited to, plates, rods, bars, irregular shapes, etc., are suitable for use in the present invention. As will be appreciated by those skilled in the art, the composition, shape, size, and/or density of the particles may widely vary.

In some instances, it is desired to modify the probes so that they are more readily able to bond specifically with the analyte. The probes can be modified with certain specific binding species to form conjugated probes. Specific binding species generally refer to a member of a specific binding pair, i.e., two different molecules where one of the molecules chemically and/or physically binds to the second molecule. For instance, immunoreactive specific binding species include antigens, haptens, aptamers, antibodies, and complexes thereof, including those formed by recombinant DNA methods or peptide synthesis. An antibody can be a monoclonal or polyclonal antibody, a recombinant protein or a mixture(s) or fragment(s) thereof, as well as a mixture of an antibody and other specific binding members. Other common specific binding pairs include but are not limited to, biotin and avidin, biotin and streptavidin, antibody-binding proteins (such as protein A or G) and antibodies, carbohydrates and lectins, complementary nucleotide sequences (including label and capture nucleic acid sequences used in DNA hybridization assays to detect a target nucleic acid sequence), complementary peptide sequences including those formed by recombinant methods, effector and receptor molecules, hormone and hormone binding protein, enzyme cofactors and enzymes, enzyme inhibitors and enzymes, and the like. Furthermore, specific binding pairs can include members that are analogs of the original specific binding member. For example, a derivative or fragment of the analyte, i.e., an analyte-analog, can be used so long as it has at least one epitope in common with the analyte.

The specific binding species can be attached to the probes using a variety of well-known techniques. For instance, covalent attachment of the specific binding species to the probes (e.g., microparticles) can be accomplished using carboxylic, amino, aldehyde, bromoacetyl, iodoacetyl, thiol, epoxy and other reactive or linking functional groups, as well as residual free radicals and radical cations, through which a protein coupling reaction can be accomplished. A surface functional group can also be incorporated as a functionalized co-monomer because the surface of the microparticle can contain a relatively high surface concentration of polar groups. In addition, although microparticle probes are often functionalized after synthesis, in certain cases, such as poly(thiophenol), the microparticles are capable of direct covalent linking with a protein without the need for further modification. For example, in one embodiment, the first step of conjugation is activation of carboxylic groups on the particle surface using carbodiimide. In the second step, the activated carboxylic acid groups are reacted with an amino group of an antibody to form an amide bond. Besides covalent bonding, other attachment techniques, such as physical adsorption, may also be utilized in the present invention.

A variety of lateral flow assay devices may be constructed in conjunction with a time-resolved up-converting luminescence detection reader. As shown in FIG. 1, a sample containing an analyte is first applied to the sampling pad. The test sample can then travel to the conjugate pad 13, where the analyte mixes with conjugated probes 40 to form analyte complexes. In one embodiment, the conjugated probe 40 is formed from microparticles that are encapsulated with a lanthanide chelate, and bound to a specific binding species for the analyte. Moreover, because the conjugate pad 13 is in fluid communication with the porous membrane 10, the complexes can migrate from the conjugate pad 13 to a detection zone 20 present on the porous membrane 10.

The detection zone 20 may contain an immobilized binding species 30 that is generally capable of forming a chemical or physical bond with the probes. For example, the binding species 30 can contain a biological binding species. Such binding species can include, but are not limited to, antigens, haptens, antibodies, protein A or G, avidin, streptavidin, secondary antibodies, and complexes thereof. It is desired that these biological binding species are capable of binding to another specific binding species (e.g., antibody) present on microparticles.

These binding species can bind and capture specifically the binding sites for probe conjugate/analyte complexes. For instance, the analytes, such as antibodies, antigens, etc., have two binding sites. Upon reaching the detection zone 20, one of these binding sites is occupied by the specific binding species of the complexed probes. However, the free binding site of the analyte can bind to the immobilized binding species. Upon being bound to the immobilized binding species, the complexed probes form a new ternary sandwich complex.

Although the detection zone 20 may indicate the presence of an analyte, it is often difficult to quantify the analyte using only a detection zone 20. Thus, the assay device may also include a calibration zone 21. The calibration zone 21 is formed on the porous membrane 10 and is positioned downstream from the detection zone 20. The calibration zone 21 is provided with a binding species 31 that is capable of binding to any remaining uncaptured conjugated probes 40 that pass through the detection zone. At the calibration zone 21, these uncaptured conjugated probes then bind to the binding species in the calibration zone. The binding species in the calibration zone 21 may be the same or different than the binding species in the detection zone 21. Moreover, similar to the detection zone 20, the calibration zone 21 may also provide any number of distinct calibration regions in any direction so that the concentration of a particular analyte within a test sample can be more accurately determined. Each region may contain the same binding species, or may contain different binding species for capturing different conjugated probes.

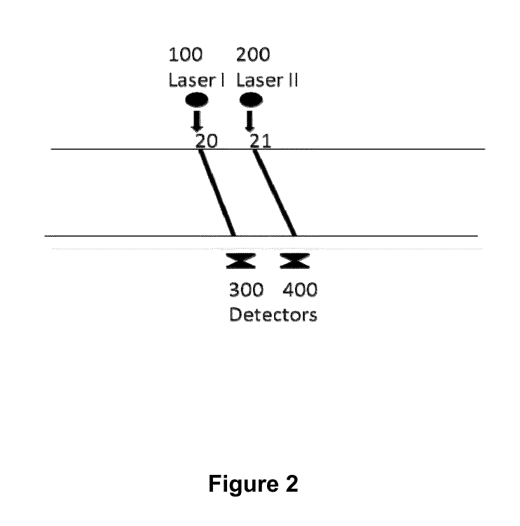

Once captured, the time-resolved up-converting luminescence signal of the conjugated probes at the detection zone 20 and calibration zones 21 can be measured using a time-resolved up-converting luminescence reader. The time-resolved up-converting luminescence reader can be constructed in many different ways. FIG. 2 shows one embodiment of the time-resolved up-converting luminescence reader that is constructed to measure the time-resolved up-converting luminescence signals from the detection zone and calibration zone in transmissional mode. The supporting material 11 of the lateral flow device for this transmissional mode measurement has a transmittance of more than 70% for upconverted visible fluorescence. The light source 100 and 200 provide pulsed near IR light or IR excitation light simultaneously onto the detection and calibration zones 20 and 21, respectively to excite the captured up-converting luminescence probes from the side of the porous membrane 10. The detector 300 and 400 are located from the opposite side of the lateral flow device, aligned with the detection 20 and 21, respectively, to measure the up-converting visible luminescence signals from the captured probes in a gated manner. Typically, the luminescence reader utilizes one or more pulsed excitation sources, preferably near IR and IR LEDs and lasers, and photodetectors that are in communication with each other and other optional components, such as optical filters. The use of pulsed excitation and time-gated detection, optionally combined with optical filters, allows for specific detection of the upconverted luminescence from only the upconverted luminescence probes, rejecting emission from other species present in the sample that are typically shorter-lived.

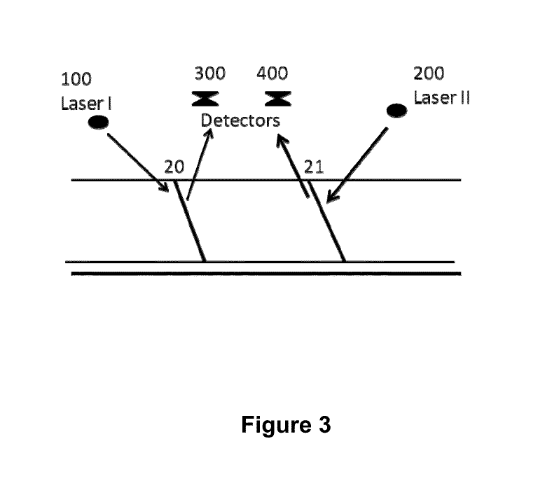

FIG. 3 shows another embodiment of the time-resolved up-converting luminescence reader that is constructed to measure the time-resolved up-converting luminescence signals from the detection zone and calibration zone in reflectance mode. The supporting material 11 of the lateral flow device for this reflectance mode measurement may have a transmittance of less than 10% for unconverted visible fluorescence. The light source 100 and 200 provide pulsed near IR light or IR excitation light simultaneously onto the detection and calibration zones 20 and 21, respectively to excite the captured up-converting luminescence probes from the side of the porous membrane 10. The detector 300 and 400 are located from the same side of the lateral flow device, aligned with the detection 20 and 21, respectively, to measure the up-converting visible luminescence signals from the captured probes in a gated manner.

Regardless of the construction of the reader, the amount of the analyte can be measured by correlating the luminescence signal, Iₛ, of the probes captured at the detection zone 20 to a calibration curve. The intensity signal, Iₛ, may also be compared with the luminescence intensity signal, I_{c}, of the probes captured at the calibration zone 21. The luminescence intensity signal Iₛ, can be compared to the luminescence intensity signal I_{c}. The total amount of the probes at the calibration zone 21 is predetermined and known and thus can be used for calibration purposes. For example, in some embodiments (e.g., sandwich assays), the amount of analyte is directly proportional to the ratio of Iₛ to I_{c}. In other embodiments (e.g., competitive assays), the amount of analyte is inversely proportional to the ratio of Iₛ to I_{c}. Based upon the intensity range in which the detection zone 20 falls, the general concentration range for the analyte may be determined. As a result, calibration and sample testing may be conducted under approximately the same conditions at the same time, thus providing reliable quantitative or semi-quantitative results, with increased sensitivity.

If desired, the ratio of Iₛ to I_{c} may be plotted versus the analyte concentration for a range of known analyte concentrations to generate a calibration curve. To determine the quantity of analyte in an unknown test sample, the signal ratio may then be converted to analyte concentration according to the calibration curve. It should be noted that alternative mathematical relationships between Iₛ and I_{c} may be plotted versus the analyte concentration to generate the calibration curve. For example, in one embodiment, the value of Iₛ/(Iₛ+I_{c}) may be plotted versus analyte concentration to generate the calibration curve.

As indicated above, sandwich formats, competitive formats, and the like, may be utilized for the lateral flow device. Sandwich assay formats typically involve mixing the test sample with antibodies to the analyte. These antibodies are mobile and linked to a label or label, such as dyed latex, a colloidal metal sol, or a radioisotope. This mixture is then contacted with a chromatographic medium containing a band or zone of immobilized antibodies to the analyte. The chromatographic medium is often in the form of a strip resembling a dipstick. When the complex of the analyte and the labeled antibody reaches the zone of the immobilized antibodies on the chromatographic medium, binding occurs and the bound labeled antibodies are localized at the zone. This indicates the presence of the analyte. This technique can be used to obtain quantitative or semi-quantitative results.

In a competitive assay, the label is generally a labeled analyte or analyte-analogue that competes for binding of an antibody with any unlabeled analyte present in the sample. Competitive assays are typically used for detection of analytes such as haptens, each hapten being monovalent and capable of binding only one antibody molecule.

Although various embodiments of device configurations have been described above, it should be understood, that a device of the present invention may generally have any configuration desired, and need not contain all of the components described above.

The present invention may be better understood with reference to the following examples.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

### Example 1

### Preparation of Up-Converting Luminescence Probe

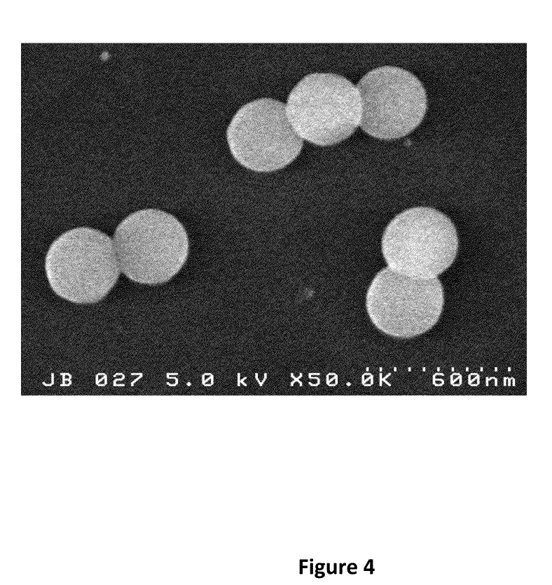

An appropriate amount of (200 μl) carboxylic acid-functionalized latex particles (0.33 μm in diameter) in aqueous solution is added with a certain amount of ethanol to reach 65% of the total solvent under stirring. The particle suspension is slowly added with an appropriate amount (e.g., 1% weight of the latex particles) of a proprietary europium chelate in ethylene chloride (e.g., 1% weight of the total solvents) under stirring. The mixture is stirred for half hour. Then a proper amount of water (e.g., three times amount of the total initial solvents) is slowly added to the stirring mixture over a certain period of time (e.g., 2 hours). After completing the addition of water, most of the ethanol in the mixture is removed through a rotovapor. The particles are then washed twice by 90% ethanol through centrifugation. The particles are then washed twice with water. The washed particles are then suspended by sonication in tris buffer containing 0.5% Tween 20. FIG. 4 shows the scanning electron micrograph of the particles.

### Example 2

### Conjugation of Antibody to the Up-Converting Luminescence Probes

The carboxylic acid surface groups of probes prepared in example 1 are first activated by carbodiimide using a standard method. Anti-biotin antibody is mixed with the activated probes for four hrs. The probes are washed four times by Hepes buffer and suspended in Hepes buffer containing 10 mg/ml BSA and 0.5% Tween 20 for storage.

### Example 3

### Time-Resolved Up-Converting Excitation and Emission Spectra of the Conjugates Prepared in Example 2

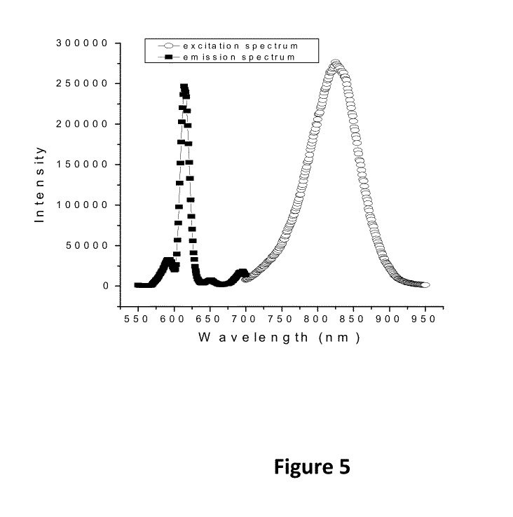

An appropriate amount of the conjugates is suspended in water to make a particle suspension in a cell (e.g., concentration to be 10 ng/cell). The time-resolved up-converting excitation and fluorescence spectra are shown in FIG. 5 by using the following measuring parameters. For time-resolved up-converting fluorescence spectrum: excitation at 870 nm, sample window at 50 μs, time-per-flash at 100 μs, initial delay at 0.01 μs, number of flash at 10, and number of scan at 10, fluorescence collection from 500 nm to 800 nm. For time-resolved up-converting excitation spectrum: emission at 615 nm, sample window at 50 μs, time-per-flash at 100 μs, initial delay at 0.01 μs, number of flash at 10, and number of scan at 10, excitation collection from 700 to 900 nm as shown in FIG. 5.

### Example 4

### Fluorescence Decay of Up-Converting Fluorescence of the Conjugates

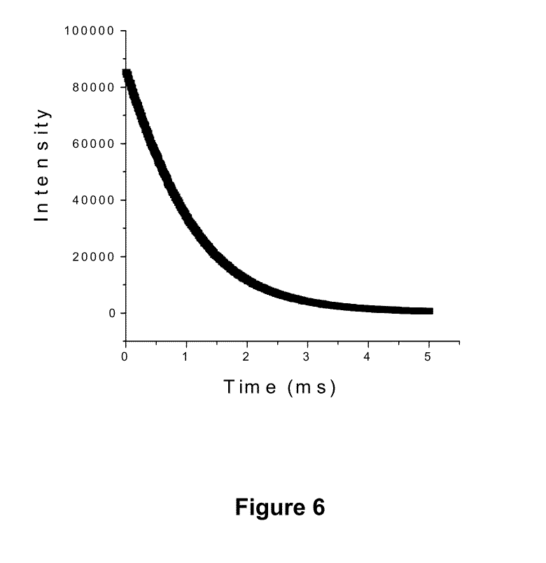

An appropriate amount of the conjugates is suspended in water to make a particle suspension in a cell (e.g., concentration to be 10 ng/cell). The decay is shown in FIG. 6. The following parameters are used to measure the fluorescence decay: excitation at 870 nm, emission at 615 nm, sample window at 50 μs, time-per-flash at 100 μs, initial delay at 0.01 μs, number of flash at 10, and number of scan at 10.

### Example 5

### Preparation Biotinylated b-Casein

An appropriate amount of b-casein (e.g., 500 mg) in borate buffer is added with a proper amount (e.g., 300 mg) of NHS-LC-biotin in borate buffer. The mixture is allowed to react for a few hours. After reaction, dialysis is used to separate biotin-conjugated b-casein from the non-conjugated biotin in PBS buffer.

### Example 6

### Preparation of Lateral Flow Devices

Nitrocellulose membrane on a plastic support card is striped with a line of 10 mg/ml biotin-conjugated b-casein in water to form a detection zone. The membrane card is dried at room temperature. A cellulose wicking pad is laminated to the end of the nitrocellulose membrane with a 4 mm overlap with the nitrocellulose membrane. The card is cut into 6 mm wide strips.

### Example 7

### Quantifying the Conjugates Captured by the Detection Zone

To each of six wells in a micro-titer plate is added with a different amount of the conjugated luminescent probe made in Example 2, ranging from 0, 5, 20, 50, 100 and 200 ng in 60 μl of 50 mM PBS buffer (pH: 7.2), containing 0.1% Tween 20 and 5 mg/ml BS, for well 1, 2, 3, 4, 5, and 6, respectively. To each well is inserted with a lateral flow device made in Example 6. After 20 minutes, the lateral flow devices are allowed to air-dry for 30 minutes. The time-resolved up-converting luminescence at 615 nm on the detection zone of each lateral device is measured at 20 us delay by exciting the detection zone using 870 nm pulsed lights. The intensity of the delayed up-converting luminescence at 615 nm is 7, 11, 19, 39, 80, and 170, respectively.

1. A lateral flow assay device for detecting and quantifying an analyte, comprising: (1) a porous membrane laminated on a solid support material, and the porous membrane has a detection zone; (2) a first specific binding species immobilized on the detection zone; (3) a conjugate pad that is in fluid communication with the porous membrane and is deposited with detection conjugate probes; wherein the detection conjugate probes have up-converting luminescence probes modified with a second specific binding species.

2. A lateral flow assay device for detecting and quantifying an analyte, comprising: (1) a porous membrane laminated on a solid support material, wherein the supporting material is substantially transparent to visible light, and the porous membrane comprising a detection zone and a calibration zone physically separated along a liquid flow direction; (2) a first specific binding species immobilized on the detection zone; (3) a second specific species immobilized on the calibration zone; (4) a conjugate pad that is in fluid communication with the porous membrane and is deposited with a detection conjugate and a calibration conjugate that are releasable upon in contact with a liquid sample, wherein the detection conjugate and calibration conjugate have an up-converting fluorescence probes modified with a third and fourth specific binding species, respectively; (5) a wicking pad that collects the fluid that passes through the detection and calibration zone.

3. The lateral flow assay device of claim 1 further comprising a calibration zone physically separated from the detection zone along the liquid flow direction, where the detection zone with a third specific binding species.

4. The lateral flow assay device of claim 1 further comprising a wicking pad that collects the fluid that passes through the detection and calibration zone.

5. The lateral flow assay device of claim 1 wherein the conjugate pad contains releasable calibration conjugate probes which have up-converting luminescence probes modified with a fourth specific binding species

6. The lateral flow assay device of claim 1 wherein the supporting material is substantially transparent to visible light with a transmittance of more than 70%

7. The lateral flow assay device of claim 1 wherein the supporting material is opaque to visible light with a transmittance of less than 10%

8. The detection conjugated probes in claim 1 are micro- and nano-particles that generate a luminescence in visible region with an emission lifetime of greater than about 10 microseconds when excited by near IR and IR photons.

9. The detection conjugated probes of claim 1 emit luminescence of higher energy than the excitation photons.

10. The detection conjugate probes of claim 1 emit luminescence with a peak at more than 50 nm shorter than the wavelength of the excitation light.

11. The micro- and nano particles of claim 7 are encapsulated with lanthanide chelate of samarium, dysprosium, europium, terbium, or combinations thereof.

12. The micro- and nano particles of claim 7 are doped nanocrystals of lanthanide of samarium, dysprosium, europium, terbium, or combinations thereof.

13. The detection zone of the lateral flow assay device in claim 1 comprising multiple detection regions.

14. The detection regions of claim 12 further comprising multiple capture reagents for binding to multiple analytes.

15. The specific binding species of claim 1 are an antigen or antibody.

16. The specific binding species of claim 2 are an antigen or antibody.

17. A method for detecting the presence or quantity of an analyte residing in a test sample comprising the following steps:
i) providing a lateral flow assay device that comprises a porous membrane in fluid communication with a conjugate medium, the conjugate medium containing up-converting luminescence probes modified with a first specific binding species configured to bind with the analyte and the said probes having a luminescence emission lifetime of greater than about 1 microsecond in the visible region when excited by a pulsed near IR or IR light source, said porous membrane defining a detection zone within which is immobilized a second binding species configured to bind with the analyte, and wherein the porous membrane defines a calibration zone positioned downstream from the detection zone within which is immobilized a third specific binding species configured to bind with the probes;
ii) contacting the conjugate probes with the test sample and allowing the probes to flow to said detection zone and said calibration zone;
iii) subjecting the detection zone to pulses of near IR or IR illumination to generate a detection signal in the visible region and, after a certain period of time has elapsed following a pulse, measuring the intensity of the detection signal, wherein a luminescence reader is employed to provide the illumination and measure the intensity of the detection signal, the reader comprising a pulsed near IR or IR excitation source and a time-gated detector for luminescence in visible region;
iv) subjecting the calibration zone to pulses of near IR or IR illumination to generate a calibration luminescence signal in visible region and after a certain period of time has elapsed following a pulse, measuring the intensity of the calibration signal; and
v) comparing the intensity of the detection signal to the intensity of the calibration signal, wherein the amount of the analyte within the test sample is proportional to the intensity of the detection signal as calibrated by the calibration signal.

18. The method of claim 16, wherein the detection zone and the calibration zone are simultaneously subjected to pulses of illumination.

19. The method of claim 16, wherein the intensity of said detection signal and the intensity of said calibration signal are measured simultaneously.

20. The method of claim 16, wherein the intensity of the detection signal is measured after a certain period of time has elapsed following each pulse.

21. The method of claim 16, wherein the intensity of the detection signal is measured after about 20 to about 200 microseconds.

22. The method of claim 16, wherein the intensity of the calibration signal is measured after a certain period of time has elapsed following each pulse.

23. The method of claim 16, wherein the intensity of the calibration signal is measured after about 20 to about 200 microseconds.
